(19) United States
(12) Patent Application Publication
(10) Pub. No.: US 2006/0286086 A1
(43) Pub. Date: Dec. 21, 2006

(54) Lysozyme-based food stuff

(75) Inventors: Stefano Ferrari, Rhinebeck, NY (US); Seth Feuerstein, Woodbridge, CT (US)

Correspondence Address: Norris, McLaughlin & Marcus P.A., 18th Floor, 875 Third Avenue, New York, NY, 10022, US

(73) Assignee: Saint Simeon LDA, Rhinebeck, NY (US)

(21) Appl. No.: 11/157,677

(22) Filed: Jun. 21, 2005

(51) Int. Cl. A61K 38/47; A61K 47/00

(52) U.S. Cl. 424/94.61; 424/439

(57) ABSTRACT

Homogenized, semi-solid food stuff fortified with lysozyme for augmentation of the immune system and to aid the immune compromised.

### BACKGROUND OF THE INVENTION

1. Field of the Invention

The present invention relates generally to a homogenized, semi-solid food stuff fortified with lysozyme

2. Description of Related Art

It is of growing concern that people, especially persons with immunity-compromised systems show due care with foods they consume. For example, the elderly and the young have immune systems which are compromised. The elderly are more susceptible to afflictions that come with increasing age. Infants have immune systems that are still developing and maturing. In fact, a study has shown food poisoning in infant food wherein strains of Bacillus licheniformis were found. (See Salkinoja-Salonen, M. S. et al., Toxigenic Strains of Bacillus licheniformis Related to Food Poisoning, Applied and Environmental Microbiology, October 1999, p. 4637-4645, Vol. 65, No. 10). For infants, breastmilk contains many of the nutrients required for development and augmentation of the immature immune system. Breastmilk also contains non-nutritional components that may promote infant health, growth and development, such as antimicrobial factors, digestive enzymes, hormones, trophic factors, and growth modulators. However, it is not always possible to obtain food, such as breastmilk, for those people whose immunity system is compromised. In addition, liquid food such as breastmilk may not always be available or be optimal for the circumstances. On the other hand, solid food is not necessarily appropriate for infants and the elderly, because they may not be able to chew the food properly and/or may choke on the food. Therefore, there is a need to protect people whose immune system is compromised from infections, for example, by producing food which contains elements which are helpful to combat bacteria, virus, fungi, or other microbial particles which reside in the food. At the same time, there is a need to produce food which is more easily consumable by people such as infants and the elderly. Examples include U.S. Pat. No. 6,270,827 and U.S. Pat. No. 6,020,015 which discloses a synthetic infant formula composition based on human milk proteins or host resistance factors, wherein one of the human milk proteins is lysozyme. However, both U.S. Pat. No. 6,270,827 and U.S. Pat. No. 6,020,015 are limited to solid and/or liquid forms.

Lysozyme is well known as an effective immunological agent. It is widely used in human therapy for the treatment of viral and bacterial infections. For example, U.S. Pat. No. 5,041,236 discloses an antibacterial composition using a ruminant stomach lysozyme. A further example is found in U.S. Pat. No. 4,355,022, which discloses an antibacterial solution comprising lysozyme for the oral cavity. Lysozyme is found in a large number of animal fluids, such as tears, pleural fluid, saliva, human milk and blood serum, as well as in a variety of organs such as the kidneys and lungs. In human milk, for example, lysozyme is present in a concentration ranging from about 0.12 g/L to about 0.5 g/L. Lysozyme is defined as a 1,4-beta-N-acetylmuramidase which cleaves the glycoside bond between the C-1 of N-acetyl-muramic acid and C-4 of N-acetylglucosamine in the peptioglycan of bacteria (See Phillips, D. C., The three-dimensional structure of an enzyme molecule, Sci. Am. 215: 78-90, 1966). The protective role of lysozyme has been observed to include lysis of microbial cell walls, adjuvant activity of the end products of peptidoglycan lysis, direct immunomodulating effects on leukocytes, and neutralization of bacterial endotoxins. Lysozyme is effective against gram positive and gram negative bacteria, as well as some types of yeasts. In this capacity, lysozyme can function as a broad spectrum antimicrobial agent. (See generally Biggar, W. D. and Sturgess, Role of lysozyme in the microbicidal activity of rat alveolar macrophages, J. M., Infect. Immunol. 16: 974-982, 1977); Thacore, M. and Willet, H. P., The formation of spheroplasts of Mycobacterium tuberculosis in tissue culture cells, Am. Rev. Resp. Diseases, 93: 786-790, 1966)). The antimicrobial effects of lysozyme often act synergistically with other defense molecules, including immunoglobulin and lactoferrin (See Jouriex et al., Protides of the Biological Fluids, Proc. 31^{st} Coll., 1984).

Because lysozyme is a natural constituent of the hen egg white, it is viewed as completely harmless as an ingredient in food. The current commercial source for lysozyme is from hen egg whites. The human and hen egg white forms of lysozyme have 60% sequence similarity, but have a very similar 3-dimensional structure. Human and hen egg white lysozymes differs in the amino acid sequence by 51 of 129 residues with one insertion at the position between 47 and 48 in the hen lysozyme (See Mine, Shouhei et al. Analysis of the internal motion of free and ligand-bound human lysozyme by use of ¹⁵N NMR relaxation measurement: A comparison with those of hen lysozyme. Protein Science 9: 1669-1684, 2000). Recently, it was disclosed in U.S. 2004-0111766 A1 a process for isolating a recombinant form of human lysozyme from a transgenic rice plant. In fact, U.S. Pat. No. 6,270,827 employs human lysozyme due to the alleged side effects due to a body's potential immune response when hen egg white lysozyme is used for medical purposes. However, studies have shown that immunogenic reactions to the hen egg white form of lysozyme, if any, are minor. Antibody reactions have been seen to other protein components in the egg white, but rarely to lysozyme. (See Langeland, T. & K. Aas, Allergy to Hen's Egg White; Clinical and Immunological aspects. In: Brostoff, J. & S. J. Challacombe (eds.) Food Allergy and Intolerance, London, Bailliere Tindall, 1987, pgs. 367-374). U.S. Pat. No. 6,020,015 and U.S. Pat. No. 6,270,827 disclose synthetic infant formula compositions based on human milk proteins. However, both U.S. Pat. No. 6,270,827 and U.S. Pat. No. 6,020,015 are limited to solid and/or liquid forms. Both U.S. Pat. No. 6,270,827 and U.S. Pat. No. 6,020,015 disclose an infant formula in liquid or concentrate form. In addition, they do not disclose lysozyme in combination with trypsin. The isolated lysozyme in U.S. 2004-0111766 A1 discloses use in food, but again, the lysozyme is for use in solid or liquid form. Hen egg white lysozyme is employed in GB 2 379 166 A, which discloses animal feed. The animal feed is for monogastric and/or non-ruminant animals such as poultry, pigs, piglets, calves and fish. There is no disclosure of the use of a human recombinant lysozyme or of the animal feed containing lysozyme in combination with lactoferrin and/or trypsin.

Therefore, there is a need for a food stuff fortified with lysozyme, especially for those people whose immunity systems are compromised, without the foregoing disadvantages. The present invention solves the deficiencies stated in the prior art, while providing improvements as stated herein.

### SUMMARY OF THE INVENTION

The present invention relates generally to a homogenized, semi-solid food stuff fortified with lysozyme.

### DETAILED DESCRIPTION OF THE INVENTION

Lysozymes act as enzymes that cleave peptidoglycans, a ubiquitous cell wall component of microorganisms, in particular bacteria. Gram-positive bacteria are highly susceptible to lysozyme due to the polypeptidoglycan on the outside of the cell wall. Gram-negative strains have a single polypeptidoglycan layer covered by lipopolysaccharides and are therefore less susceptible to lysis by lysozyme.

Lysozyme from human and non-human sources is contemplated. In one example, U.S. 2004-011766, which is hereby incorporated by reference, discloses human recombinant lysozyme which is expressed in rice. In another example, isolated human lysozyme is disclosed in U.S. Pat. No. 5,618,712, which is hereby incorporated by reference. Lysozyme has been isolated and/or reported in non-human sources ranging from the hen egg white (U.S. Pat. No. 3,515,643, which is hereby incorporated by reference) to the ficus plant (See Meyer, K. et al, Lysozyme of Plant Origin, J. of Biol. Chem., 1946, Vol. 163., Pages 733-740) to the Asterias rubens, or common starfish (Bachali, Sana, et al., The lysozyme of the starfish Asterias rubens, 2004, Eur. J. Biochem., Vol. 271, Pages 237-242).

Lactoferrin comprises a protein found naturally within biological fluids, such as milk and saliva, at mucosal surfaces and within white blood cells. It is thought that lactoferrin has anti-bacterial properties, while still protecting the body. In addition, lactoferrin appears to effectively kill a range of fungi and yeasts, including the causative agent of thrush, Candida albicans. Moreover, research has shown that lactoferrin can prevent viruses, such as HIV, hepatitis and CMV, from binding to the body's cells and therefore prevents viral infection.

Lactoferrin is one of the principle proteins responsible for providing protection to infant mammals before their immune systems begin to function. It is a minor protein in cow's milk (0.3% by weight) and is extracted from skim milk or whey through protein separation. Apart from milk, lactoferrin is generally produced and released in the body in the digestive, respiratory and reproductive systems through bodily secretions such as saliva, tears, and nasal secretions. Lactoferrin is also produced by a special group of white blood cells known as neutrophils.

Lactoferrin occurs naturally in three forms: (i) iron-saturated, (ii) iron-free, and (iii) immobilized (Activated). It is thought that the iron-free and immobilized forms of lactoferrin have the highest antimicrobial abilities through the binding of iron required by bacteria for growth and the ability for lactoferrin to detach bacteria from surfaces and eliminate bacterial attachment structures.

In a preferred embodiment, the homogenized, semi-solid food stuff is fortified with lysozyme and/or lysozyme in combination with lactoferrin and/or trypsin. The term “fortified” as used herein means 1 to 4 times the concentration normally found in human breastmilk. Trypsin is a digestive enzyme produced in the pancreas to digest proteins. It has been used in treatments for wounds and for diabetes. It has also been used in food processing in infant formulas to aid in digestion. In one preferred embodiment, the homogenized, semi-solid food stuff is fortified with both lysozyme and trypsin. In another preferred embodiment, the homogenized, semi-solid food stuff is fortified with lysozyme, lactoferrin and trypsin. In a preferred embodiment, the lysozyme is either isolated from the hen egg white or in the human recombinant form.

The homogenized, semi-solid food stuff can be any food stuff that is generally consumed by infants, toddlers, young children, geriatric patients or persons whose immunity systems are compromised such as HIV patients, cancer patients and transplant patients. The homogenized, semi-solid food stuff can be combined with other food stuffs such as diabetic formulas. “Young children” as used herein comprises children that are between the age of over 3 years old to less than 13 years old. “Semi-solid” as used herein excludes liquids such as synthetic infant milk formulas and solids such as powders. In one preferred embodiment, the homogenized, semi-solid food stuff is a pureed food. In one preferred embodiment, the homogenized, semi-solid food stuff which is fortified with lysozyme has a content of lysozyme which is about 0.25 g/L to about 2.0 g/L, and more preferably about 1.0 g/L to about 2.0 g/L.

It should be understood that the preceding is merely a detailed description of one preferred embodiment or a small number of preferred embodiments of the present invention and that numerous changes to the disclosed embodiments can be made in accordance with the disclosure herein without departing from the spirit or scope of the invention. The preceding description, therefore, is not meant to limit the scope of the invention in any respect. Rather, the scope of the invention is to be determined only by the issued claims and their equivalents.

What is claimed is:

1. A homogenized, semi-solid food stuff fortified with lysozyme.

2. The homogenized, semi-solid food stuff according to claim 1, which comprises lactoferrin and/or trypsin.

3. The homogenized, semi-solid food stuff according to claim 1, wherein the lysozyme is a human recombinant lysozyme.

4. The homogenized, semi-solid food stuff according to claim 1, wherein the lysozyme is a non-human lysozyme.

5. The homogenized, semi-solid food stuff according to claim 4, wherein the non-human lysozyme is a hen egg white lysozyme.

6. The homogenized, semi-solid food stuff according to claim 1, wherein the lysozyme is at a concentration of about 0.25 to about 2.0 g/L.

7. The homogenized, semi-solid food stuff according to claim 6, wherein the lysozyme is at a concentration of about 1.0 to about 2.0 g/L.

8. The homogenized, semi-solid food stuff according to claim 1, which is formulated for a person whose immune system is compromised.

9. The homogenized, semi-solid food stuff according to claim 7, which is formulated for infants, toddlers and/or young children.

10. The homogenized, semi-solid food stuff according to claim 1, which is formulated for geriatric patients.

11. The homogenized, semi-solid food stuff according to claim 1, which is formulated with a diabetic formula.

12. A method of protecting against infections said method comprises administering to a person an effective amount of a homogenized, semi-solid food stuff according to claim 1.

13. A method of protecting against infections in a person with a compromised immune system, said method comprises administering to the person an effective amount of a homogenized, semi-solid food stuff according to claim 1.
